(19) United States
(12) Patent Application Publication
(10) Pub. No.: US 2008/0139985 A1
(43) Pub. Date: Jun. 12, 2008

(54) Patella Femoral Brace

(76) Inventor: Robert Gilmour, Auckland (NZ)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 10/591,968

(22) PCT Filed: Mar. 10, 2005
(86) PCT No.: PCT/US05/08136

§ 371 Date: Aug. 13, 2007

(30) Foreign Application Priority Data
Oct. 3, 2004 (NZ) 2004/0531703

(51) Int. Cl. A61F 5/01; A61F 5/00

(52) U.S. Cl. 602/26

(57) ABSTRACT

A patella femoral brace having a support member configured to be worn about the knee of a user, the support member having an upper portion (10) extending above the knee and a lower portion (12) extending below the knee, and a pressure cuff (32) attachable to the support member in a position to exert force on at least a portion of the periphery of the user's patella. The pressure cuff can have a plurality of connection members (33) for securement to the sleeve (16) at selective ones of a plurality of connection points (25, 26, 27) provided on opposite sides of the knee and above and below the knee, such that the position of the pressure cuff is adjustable by varying connection of the connection members to selective ones of the connection points to apply force to the patella in a plurality of directions. A buttress (24) can be provided between the pressure cuff and the patella.

### BACKGROUND

1. Field of the Invention

The invention relates generally to orthotic devices, and more particularly to a patella-femoral brace.

2. Description of the Prior Art

Patella-femoral bracing is used to treat conditions of the patella-femoral joint. This is the joint between the patella and the anterior femoral condyle just proximal to the knee. The patella is a sesamoid bone in so far as it lies within a tendon; namely the quadriceps tendon. Its function is to act as “pulley” for the tendon as it crosses the knee joint. Because the patella lies in the tendon, its position relative to the anterior femoral surface is determined by the line of pull of the quadriceps, and by the structures which assist in holding it in the femoral groove throughout the range of motion. The most common cause of patella femoral pain is mal-tracking of the patella.

Any mal-tracking can occur during flexion or extension, more commonly in mild cases, during extension, and more commonly in severe cases during flexion.

Buttresses of various shapes have been used to help stabilise the patella. Most surround the edges of the articulating portion the patella as the superior pole or top portion does not articulate and is completely encapsulated by tendon, so a buttress can have little effect.

Most mechanisms are designed to exert a unidirectional force on the patella in the medial-lateral direction throughout knee movement such that lateral movement or “lateral drift” which leads to subluxation or dislocation is prevented.

However, the tracking of a patella in any individual can vary quite considerably. Rather than moving in a linear up and down motion or medial and lateral motion, the patella tends instead to “snake” its way in a general up and down direction throughout the range of knee motion. This is determined by the relative forces exerted by the quadriceps, the supporting structures, and the shape of an individuals articular surfaces within the patella-femoral joint.

Therefore, it is an object of the present invention to provide an orthotic device which will at least go some way toward overcoming disadvantages of existing constructions, or which will at least provide the public with a useful alternative.

### SUMMARY

According to a preferred embodiment of the invention, a patella-femoral brace is provided comprising a support member and a pressure cuff adapted to apply force to the patella to assist proper tracking of the patella during movement of the knee. The positioning of the pressure cuff against the patella is adjustable, and can be maintained in a required position relative to the patella to apply a desired force to the patella in desired direction, including in a medial-lateral plane, or direction, and an inferior-superior plane, or direction. Preferably a resultant force may be applied to the patella in any direction in the medial-lateral direction, the inferior-superior direction, or any intermediate directions.

Preferably support member can comprise an upper substantially rigid arm and a lower substantially rigid arm, the arms being hingedly interconnected so as to move rotatably relative to each other during movement of the knee. Connection members and various connection points can be utilized to attach the pressure cuff directly or indirectly to the upper arm and the lower arms in a manner which permits adjustment of the pressure cuff relative to the patella.

Preferably the support member can further comprise a flexible sleeve which supports, or is supported by, the rigid upper and lower arms, the sleeve being adapted to locate about the knee of the user.

Preferably the sleeve further comprises a buttress adapted for location about a periphery or a peripheral portion of the patella.

Preferably the cuff is located in use about an exterior surface of the buttress, so as to apply a force to the buttress, and therefore apply a force to the patella.

Preferably the buttress can include a liner on an inner surface adjacent the patella, in which the liner is a soft foam material which conforms to the particular shape of the individual user's patella when force is applied by the cuff such that an enhanced fit is provided.

Preferably the buttress has a convex outer surface and the cuff has a convex inner surface which mates with the convex buttress surface

Preferably the support member can further comprise another set of rigid upper and lower arms which are hingedly interconnected, which are provided for location on an opposite side of the knee from the first set of hinged upper and lower rigid arms.

Preferably an embodiment of the cuff according to the invention comprises at least three connection members, one connection member being connected to one side of the sleeve and two connection members being connected to the other side of the sleeve, one of the two being connected above the hinged engagement and the other being connected below the hinged engagement.

An alternative embodiment of the cuff can comprise four connection members, one connection member being connected directly or indirectly to each of the four arms.

Preferably the position of the engagement of at least some of the connection members is adjustable to thereby adjust the force applied to the patella.

Preferably the connection members comprise adjustable straps. Preferably the straps are resilient or elastic.

Preferably the arrangement of the connection members and the arms or sleeve is such that a desired force or change in force can be applied to the patella during flexion of the knee, and the force or change in force can be applied in a desired direction or range of directions.

Preferably a chafe is provided to enable each connection member to engage with an arm or sleeve.

Preferably each chafe includes a pin which is adapted to be received in an aperture provided on the arm or sleeve whereby the chafe may rotate within a range of movement relative to the arm or sleeve.

Preferably the pin includes a protrusion, or head, having a shape which is selected to allow the chafe to be engaged in the aperture in a first orientation, but to be unable to be removed from the aperture when the chafe is in a second, operational, orientation.

Alternatively, the cuff may be a relatively flexible member integrated with the sleeve, having one portion fixed to a side of the sleeve, or a rigid upper and/or lower arm at a side of the sleeve, and a pair of flexible connection members attachable at variable locations on an opposite side of the sleeve.

Preferably, one of the pair of flexible connection members is attachable above the patella and the other is attachable below the patella, such that the cuff can apply an adjustable force on the patella in the aforesaid medial-lateral direction, inferior-superior direction, and intermediate directions.

Further aspects of the invention will become apparent from the following description.

### BRIEF DESCRIPTION OF THE DRAWING FIGURES

One or more embodiments of the invention will be described with reference to the accompanying drawings in which:

FIG. 1 is a front elevation of an embodiment of a patella-femoral knee brace according to the invention;

FIG. 2 is a rear elevation view of the brace of FIG. 1;

FIG. 3 is a side elevation view of the brace of FIG. 1;

FIG. 4 is an elevation view of an embodiment of a pressure cuff according to the invention;

FIG. 5 is a front view of another embodiment of a patella-femoral brace according to the invention.

FIG. 6 is a perspective view of the brace of FIG. 7 showing the structure of the brace adjacent the inner knee.

FIG. 7 is a perspective view of the brace of FIG. 7 showing the structure of the brace adjacent the outer knee.

FIGS. 8A and 8B illustrate a further embodiment of a pressure cuff according to the invention.

FIG. 9 illustrates a still further embodiment of a pressure cuff according to the invention.

FIG. 10 is an elevation view of another embodiment of a pressure cuff;

FIG. 11 is an exploded perspective view of the pressure cuff of FIG. 5;

FIG. 12 is a perspective view of an embodiment of a four point pressure cuff according to the invention.

FIG. 13 is a further embodiment of a four point pressure cuff according to the invention.

FIG. 14 is a further embodiment of a four point pressure cuff according to the invention.

FIG. 15 is a perspective view from above of an embodiment of a connection member according to the invention;

FIG. 16 is a perspective view from below of the connection member of FIG. 15;

FIG. 17 is an exploded perspective view of an embodiment of a chafe and associated end of a rigid arm;

FIG. 18 is a perspective view of a chafe engaged upper and lower rigid arms;

FIG. 19 is a diagrammatic front elevation view of an embodiment of the invention showing a cuff engaged with pairs of upper and lower rigid arms;

FIGS. 20a-20c illustrate an embodiment of a buttress according to the invention.

FIG. 21 is a perspective view of the outer knee side of a further embodiment of a patella-femoral according to the invention.

FIG. 22 is a perspective view of an inner knee side of the brace of FIG. 21.

### DETAILED DESCRIPTION

Referring to the drawing figures, an orthotic device according to the invention, and specifically a patella-femoral brace, is shown having a support member which can comprise either, or both, upper 10 and lower 12 arms connected together by a hinge assembly 14 and a sleeve 16. The upper 10 and lower 12 arms are, in the preferred embodiment, replicated on the other side of the brace, so that a pair of arms connected by a hinge assembly is provided in use on either side of the knee of a person using the brace. Those skilled in the art will realise that the invention could be effected without replicating the arms 10, 12 and hinge assembly 14 on either side of the brace.

The arms 10 and 12 are preferably constructed from a substantially rigid material, for example an aluminium or a plastic material, or laminated materials including fiber reinforced materials or a combination of all the foregoing. The hinge assembly 14 can be constructed in a variety of different ways known to those skilled in the art to which the invention relates, and will typically be constructed using a rivet or pin to provide a pivot point that interconnects the adjacent ends of the arms and is designed to limit the range of movement.

The arms 10 and 12 are provided within an appropriate structure for engaging regions of the user's leg adjacent to the knee to enable the brace to be securely attached to the user. Therefore, in the preferred embodiment a sleeve 16 is provided which is constructed from an appropriate material such as a neoprene or elastic foam material so that the brace is securely and comfortably engaged with the user's leg within the vicinity of the user's knee. Those skilled in the art will realise that a number of different structures may be used to replace the continuous sleeve. To enable securement of the sleeve to the user, straps 18 and 20 are provided. These may include a connectable webbing material or hook and loop material such as VELCRO to facilitate tightening and engagement, but other fastening mechanisms such as buckles may be used. A region of the sleeve 22 can be replaced with a lighter material or removed altogether at the rear of the brace in order to facilitate flexion.

The sleeve 16 includes a buttress 24 which is shaped and positioned to enable it to be located in use adjacent to the edge or periphery of the articular portion of the patella. The buttress may be formed from a variety of different materials, but in the preferred embodiment it is constructed from a resilient foam or plastics material. The buttress 24 substantially surrounds the articular portion of the patella in the preferred embodiment, but could be broken or segmented, or only surround a selected portion of the articular portion of the patella.

Referring now to FIG. 3, the arms 10 or 12 (or a part of the sleeve or other structure attached to the arms) includes a number of connection points, or sockets 25, 26, 27 which are shown in more detail in subsequent drawing figures, and these may take a variety of forms. In particular, these could in some embodiments comprise Velcro attachment points or buckles. However, in the most preferred embodiment the connection points are adapted to connect with chafes provided on connection members, as will be described in more detail below.

Turning to FIG. 4 an embodiment of a cuff 32 is shown having a body 34 which is adapted to locate over the buttress 24 in use so that a force may be applied to the buttress 24. The body 34 of the cuff 32 can take a variety of forms, and in the embodiment shown in FIG. 4 is substantially “horseshoe” shaped. As shown, the cuff 32 is attached to the rigid arms 10, 12 by a plurality of connection members 33 which include chafes 30 at distal ends of the connection members 33. To use the cuff 32, a user places an attachment loop 36 over one of the hinges 14, then engages chafes 30 with a selected socket 25 to 27 of each of the upper and lower arms 10 and 12.

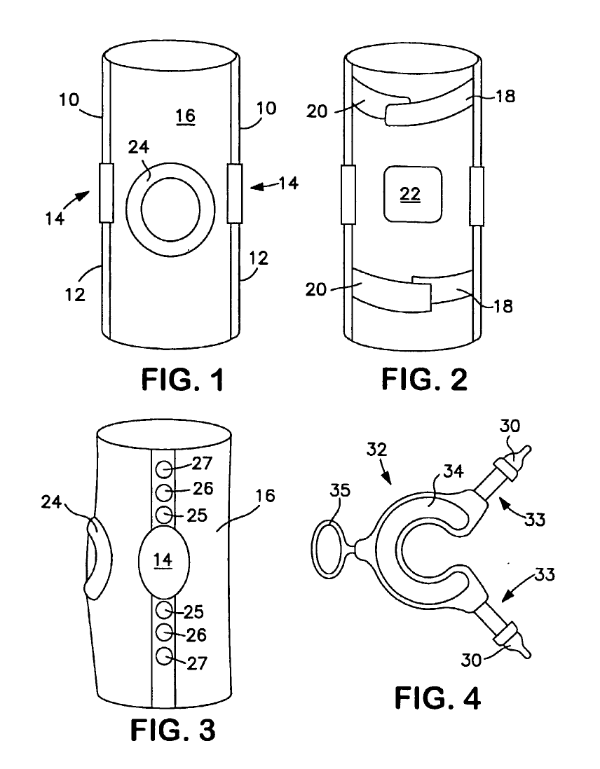

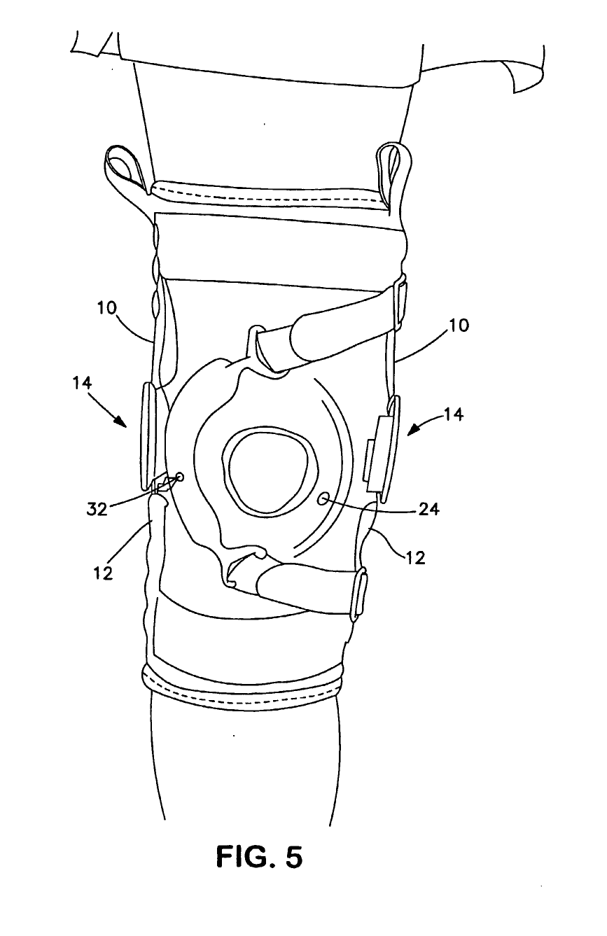

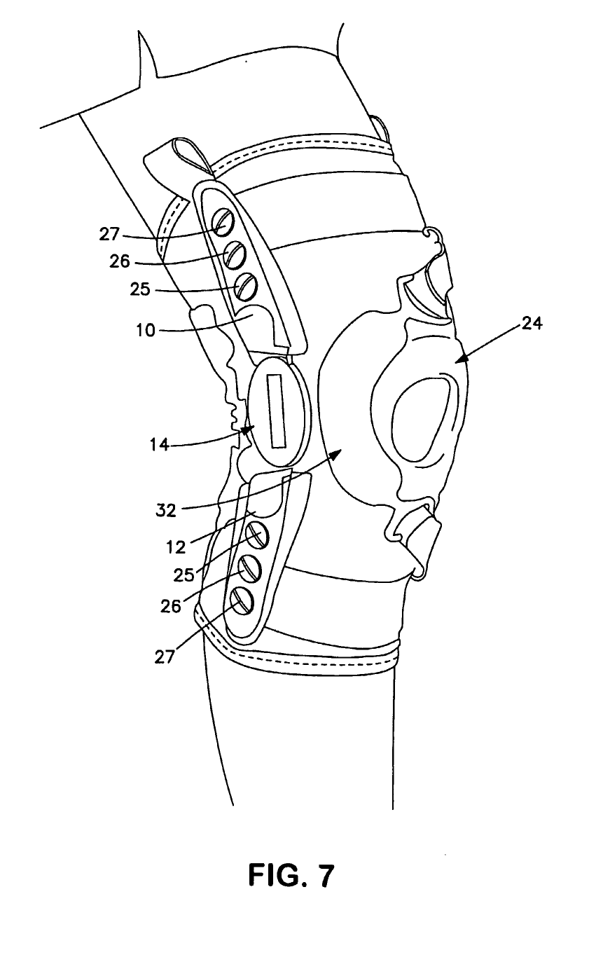

Referring to FIGS. 5, 6 and 7, a further preferred embodiment of a patella-femoral brace as shown in FIGS. 1-3 is shown with an associated pressure cuff, such as cuff 32 in FIG. 4. FIG. 7 is a front perspective view of the patella-femoral brace showing, at both sides of the brace, an upper rigid arm 10 connected to a lower rigid arm 12 by a hinge 14 which is located proximate the user's knee. A sleeve 15 is provided in combination with the pairs of upper 10 and lower 12 rigid arms, and is adapted to fit snuggly about the knee of the user. An internal buttress 24 is preferably included which is supported in place around the patella by the sleeve 15. The sleeve 15 can support, or be supported by, the pairs of hinged upper 10 and lower 12 rigid arms. In the embodiment shown, the three-point cuff 32 is attached at one side to the hinge 14 located at the outside of the knee, such as via the attachment loop 35 shown in FIG. 4. The distal ends of the cuff 32 are attached to respective opposing upper 10 and lower 12 rigid arms located at the inside of the knee, such as via connection members 33 with chafes 30 and connection points 25, 26, 27, as shown in FIGS. 3 and 4.

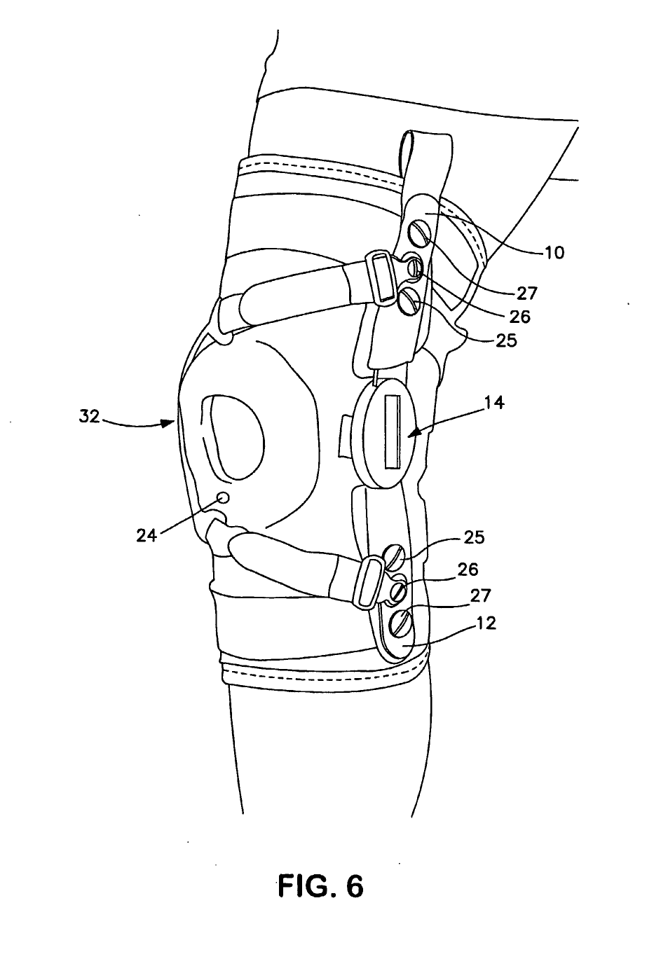

FIGS. 6 and 7 are additional views of the brace shown in FIG. 5. FIG. 6 is a perspective view of the brace structure on the side adjacent the inside of the knee, whereas FIG. 7 is a perspective view of the brace structure on the side adjacent the outside of the knee. FIG. 6 illustrates the attachment of the connection member chafes to the upper connection points, or sockets 25, 26, 27 on the upper 10 and lower 12 rigid arms at the inside of the knee. FIG. 7 illustrates attachment of the cuff 32 to the hinge 14 at the outside of the knee. Further details of preferred embodiments of connection members 33 using chafes 30 will be described in more detail hereinafter.

The pressure cuffs may be constructed from a resilient or elastic material such as a thermoplastic elastomer or thermoplastic rubber. Such material may have one or more of the following properties:

| | |
|---|---|
| Hardness | 2-45 (Shore A) |
| Specific Gravity | 0.85-0.95 (gm/cubic cm) |
| Tensile Strength | 5-60 (kg/square cm) |
| Elongation at Break | 1100-650 (%) |
| Tear Strength | 30-50 (kg/square cm) |
| Brittle Temperature | minus 30-minus 60 (degrees C.) |

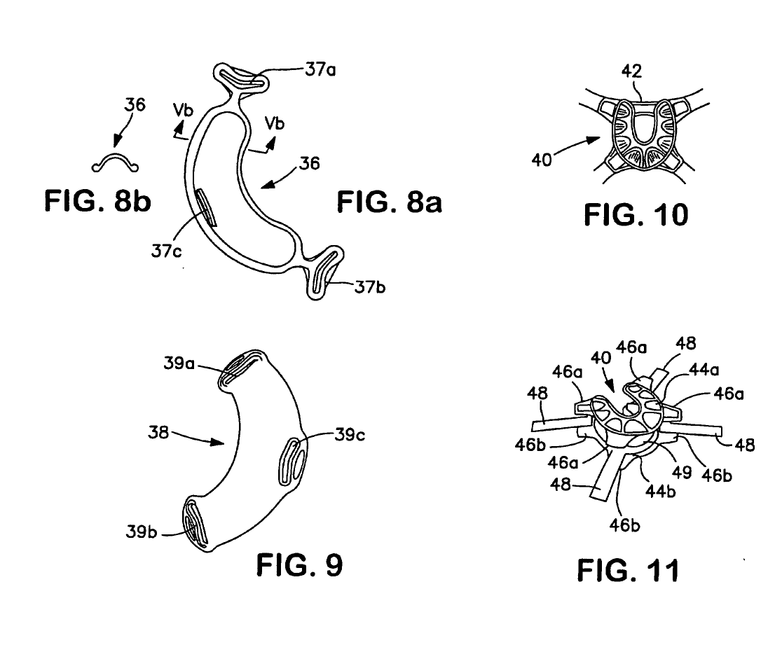

Further embodiments of pressure cuffs which can be constructed (for example by moulding) using such a material are shown, for example, below in FIGS. 8a-9 and 13-15.

An embodiment of such a pressure cuff 36 is illustrated in FIGS. 8a and 8b, comprising a relatively thin, arcuately shaped member. The cuff 36 has three attachment locations 37a, 38b, 37c by which connection members, such as connection members 33 in FIG. 4, can be utilized to attach the cuff 36 to the brace in a manner as will be described in more detail hereinafter. The cuff 36 can preferably have a concave inner curvature as shown in the cross section view in FIG. 8b.

A further embodiment of a pressure cuff 38 is illustrated in FIG. 9, also having three attachment locations 39a, 39b, 39c. The cuff 38 can otherwise be similar to the cuff 36 shown in FIGS. 8a and 8b.

Referring now to FIGS. 10 and 11, a certain preferred embodiment of a cuff 40 is shown having four connection members, or straps, 48 and each strap 48 has an engagement device such as a chafe 30 (not shown) for engagement with one of sockets 25, 26, 27 on each arm 10, 12. The cuff 40 may further include a supra patella link 42.

The body of the cuff 40 is shown in an exploded view in FIG. 11, in which it can be seen that the body includes upper 44a and lower 44b parts which include upper 46a and lower 46b tabs to which the straps 48 are connected. The upper 44a and lower 44b parts are preferably moulded to include a recess for location over an internal moulded foam structure 50, which in use is preferably welded between the cuff members 44a, 44b. One end of each of the straps 48 are connected between the upper 46a and lower 46b tabs by, for example, stitching, welding, gluing, or a combination thereof.

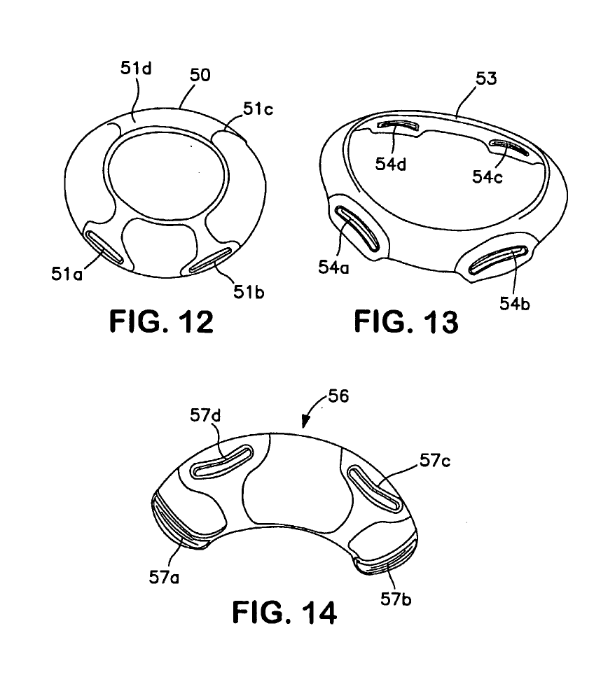

FIGS. 12, 13 and 14 illustrate certain preferred embodiments of four point pressure cuffs. A first four point pressure cuff 50 is illustrated in FIG. 12, having four attachment locations 51a, 51b, 51c, 51d, which can be equally spaced about the periphery of the cuff 50. The cuff 50 is a generally circular member that surrounds the patella and is attachable to each of the two upper 10 and two lower 12 rigid arms at the four attachment locations 54a, 54b, 54c, 54d. The attachment can be accomplished via connection members and chafes cooperating with the sockets 25, 26, 27, as described previously.

Referring to FIG. 13, another four point cuff 53 is shown, which can be similar to the cuff 50 in FIG. 12, and also includes four attachment locations 54a, 54b, 54c, 54d for engagement with connection members to attach the cuff 53 to the pairs of upper 10 and lower 12 rigid arms. The attachment locations 54a, 54b, 54c, 54d can also be generally equally spaced about the periphery of the cuff 53. The cuff 53 can be a thinner, lighter version of the cuff 50 in FIG. 12.

FIG. 14 illustrates a further embodiment of a four point pressure cuff 56 also having four attachment locations 57a, 57b, 57c, 57d. However, the cuff 56 has a semi circular shape and is designed to only encompass a periphery of the top of the patella. In use, the cuff 56 is positioned above the patella, and each of the four attachment locations are attached to each of the upper 10 and lower 12 rigid arms at either side of the knee.

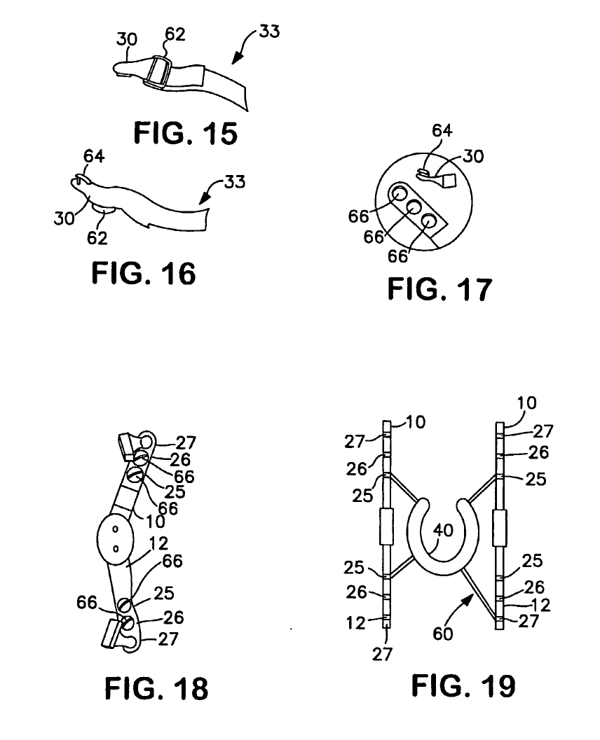

In FIGS. 15, 16 and 17, connection members, which can be straps 33 (FIG. 4) or straps 48 (FIGS. 10, 11) are shown having chafes 30 provided at distal ends thereof. The chafes 30 preferably include a buckle, or ladder lock, 62 which allows adjustment of the length of each strap. The chafe 30 also preferably includes an engagement pin having a head (64) which is shaped to engage with slots 66 provided in each socket 25, 26, 27 when the head 64 is positioned in the correct orientation.

FIG. 18 shows engagement of the head 64 with a slot 66. As can be seen from that figure, each slot 66 is arranged so that swivelling or rotational movement of the chafe 30 through the flexion/extension range of movement will not cause the chafe 30 to adopt an orientation where it can be removed from the slot 66. Accordingly, a secure engagement can be made.

According to a preferred embodiment of the brace, a user firstly engages the brace with the leg such that the internal surfaces of the buttress 24 are adjacent to the periphery of the articular portion of the patella. Then the pressure cuff 40 or 32 is placed over the buttress 24. The brace will be described with use of the pressure cuff 40 shown in FIGS. 10 and 11, however it will be seen that other embodiments of the cuff that are adjustable in multiple planes, or directions, may also be used, such as cuff 32 in FIG. 4, for example.

The attachment straps 48 of the cuff 40 are adjusted in length, and in their point of fixation to the support member, to enable the cuff 40 to be located in the correct position over the buttress 24 and to exert a desired force during the flexion/extension movement of the user's leg. In order to assist with applying an appropriate force, the straps 48 may be constructed of a resilient or elastic material.

It will be seen that the further a strap 48 is connected from the hinge 14, the longer the resultant lever arm, and thus the greater the force that will be applied as the degree of flexion increases. This concept may be used to provide considerable variation in the magnitude of the force applied to the patella by the pressure cuff. Furthermore, by selecting the degree of elasticity or resilience each connection member, greater control can be provided over the force which is applied.

For example, the straps 48 could be adjusted for maximum tension at 30 degrees of flexion, if this is the point at which it is believed that the patella deviates in its tracking. Therefore, as flexion occurs from the fully extended position, an increasing force will be applied to the patella until the 30 degree position is reached.

Therefore, if connection members that are constructed from the same material are located at the same connection points along the arm or sleeve, then the force applied to the patella will increase during flexion, but the forces will be balanced, so the resultant force will be applied a posterior direction (i.e., toward the rear of the knee).

However, if one connection member, for example, is connected to the sleeve or arm at a point further away from the hinge than the remaining connection members, then the force applied to the patella in the direction of that connection member will increase more relative to the force applied by the other connection members as flexion occurs. Accordingly, corrective forces can be customised to provide specific solution to the mal-tracking of an individual's patella.

Referring now to FIG. 19, it can be seen that the connection member indicated by arrow 60 is engaged with socket 27, whereas the remaining connection members are engaged with socket 25 of their respective arms. Therefore, as flexion occurs, there will be an increasing force applied to the patella in the direction of connection member 60 (i.e., in an inferomedial direction). It will be seen that a particular angle of flexion may be chosen at which to engage and adjust the connection members. In this way, the magnitude and direction of the applied force can be selected at that particular angle of flexion, and the adjustment may be such that as the degree of flexion increases or decreases from that selected point, the magnitude and direction of the applied force can be varied by appropriate selection of the adjustment points to which the connection members have been engaged.

In selected embodiments of the invention, the connection members, or straps, and other straps such as engagement straps 18, 20 (shown in FIG. 2), may be constructed from elasticised connectable material such as hook and loop material sold under the trademark VELCRO which has been modified by being provided with a limit to the degree of allowable elastic deformity (i.e., by limiting the degree of stretch). This maybe effected in a number of different ways. In one example, the material may be stitched in a selected pattern, such as a zigzag pattern, the angle of which is chosen so that the thread will straighten at a predetermined point and therefore that further elastic deformity. In another example, an inelastic material may be loosely attached to the webbing or hook and loop material at selected points to thereby limit the degree of deformity. It has been determined that limiting the degree of elastic deformity can be advantageous.

It will be seen that, by adjusting the points of fixation of the pressure cuff to the support member, forces of selected magnitude may be exerted on the patella in a number of different directions, for example in a medial direction, an inferior (downward) direction, or a combination of these to varying degrees. Accordingly, true multidirectional and/or multiplanar control is provided. It also be seen that, the direction and magnitude of the force may be varied depending upon the degree of flexion.

It will also be seen that the apparatus can be set up at any point in the flexion-extension cycle so that the multidirectional or multiplanar force can be made to vary over a selected range of the cycle. Therefore, a user may use the brace for specific activities which involve movements concentrated in one range of the flexion-extension cycle.

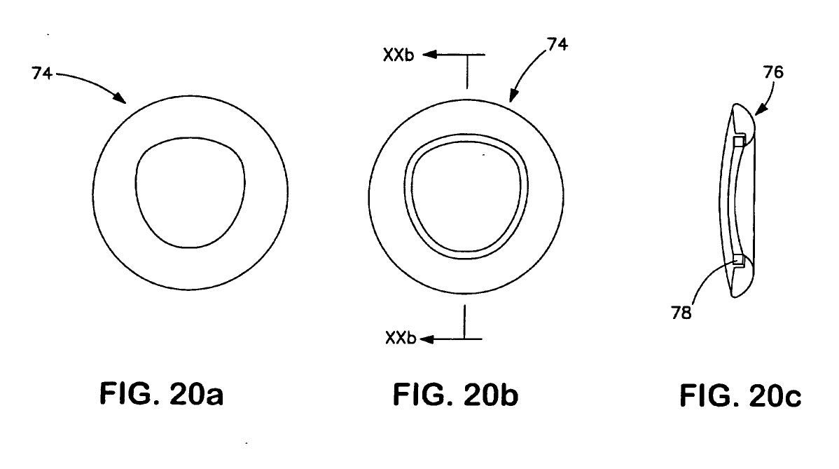

Referring to FIGS. 20a, 20b, and 20c, there is shown in the several views a preferred embodiment of a buttress 74 adapted to encircle the patella, and is preferably held in place about the patella by the sleeve 15 when the brace is worn about the knee. As shown in the cross section view in FIG. 20b, the buttress 74 preferably has a convex outer surface 76 and also preferably includes a relatively soft insert 78 located at the inner circumference of the buttress 74, for positioning adjacent the outer periphery of the patella when the brace is used. The relatively soft insert 78 can be made, for example, from a soft foam material, and can provide an enhanced grip and fit on the patella. In use, when the pressure cuff exerts a force on the buttress 74, the soft insert 78 conforms to the shape of the patella. This enables the buttress 74 to adapt more accurately to fit the specific shape of a particular person's patella profile, and therefore the buttress is essentially self-customizing.

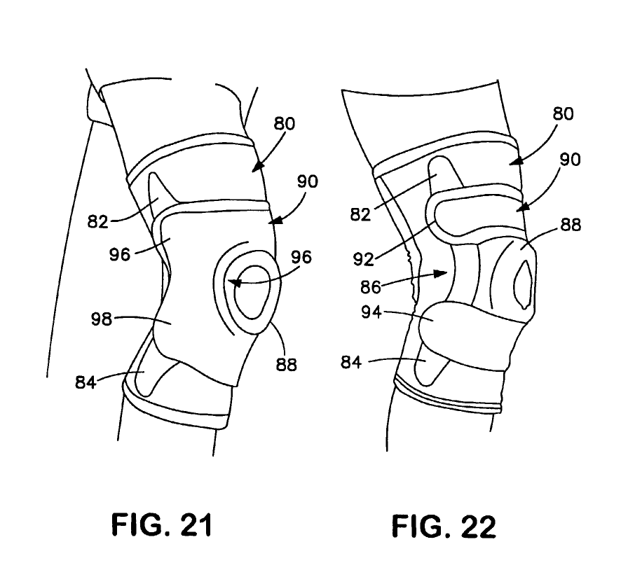

Referring now to FIGS. 21 and 22, a further embodiment of a patella-femoral knee brace is illustrated comprising a sleeve 80 and an adjustable pressure cuff 90 for exerting a force on the patella, wherein the force is adjustable and can be applied to the patella in a a plurality of directions, including a medial-lateral an inferior-superior direction, and intermediate directions. The force, and direction in which the force is applied, is adjustable by adjusting the attachment, directly or indirectly, of the pressure cuff 90 to the sleeve 80. The pressure cuff can be made integral with the sleeve, such as by fixing on side of the pressure cuff 90 to one side of the sleeve 80. Alternatively, that side of the cuff 90 could be detachable, and adjustable. The sleeve 80 preferably includes an internal buttress 88 which is supported in position surrounding the patella, intermediate the patella and the cuff 90.

The brace can further include one, or a pair of upper 82 and lower 84 rigid arms connected by hinges 86. The sleeve 80 can support, or be supported by, the upper 82 and lower 84 rigid arms.

The pressure cuff 90 in this embodiment can be made from a relatively flexible material and, as described above, can have one side of the cuff fixed to the sleeve 80 at one side of the knee. The cuff 90 further has upper 92 and lower 94 connection members extending from the fixed side of the cuff 90 side across the upper and lower periphery of the patella and are attachable to an opposite side of the sleeve 80, or upper 82 and lower 84 rigid arms. The upper and lower connection members of the cuff 90, and the junction 96, thereof form the periphery of the cuff 90 which abuts, and exerts force on, a portion of the periphery of the patella.

The attachment of the upper and lower connection members of the cuff 90 can be accomplished in the same manner described above in regard to the other embodiments of the pressure cuff, and can preferably be accomplished using a simple hook and loop fastener arrangement. The hook and loop fastener arrangement can permit adjustment in regard to the effective length of each of the upper 92 and lower 94 connection members to attach to the desired attachment location on each of the upper 82 and lower 84 rigid arms, or the sleeve 80. The cuff 90 is thus adjustable to apply force on the patella by the cuff 90 in any of the directions described above, including the medial-lateral and inferior-superior directions, as well as intermediate directions.

In the figures shown, the upper 92 and lower 94 connection members of the pressure cuff 90 extend from the side of the cuff 90 fixed to the sleeve, or rigid arms, adjacent the outside of the knee, across the knee, and are adjustably attached to the sleeve, or rigid arms, adjacent the inside of the knee. However, it should be understood that this arrangement could be reversed, such that the upper 92 and lower 94 connection members would extend from adjacent the inside of the knee to adjustable locations provided on the sleeve, or rigid arms, adjacent the outside of the knee.

Where in the foregoing description, reference has been made to specific components or integers of the invention having known equivalents then such equivalents are herein incorporated as if individually set forth.

Although this invention has been described by way of example and with reference to possible embodiments thereof, it is to be understood that modifications or improvements may be made thereto without departing from the scope or spirit of the invention.

It should be noted that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims or (included within the present invention).

What is claimed is:

1. A patella-femoral brace comprising:
a support member configured to be worn about the knee of a user, said support member having an upper portion extending above the knee and a lower portion extending below the knee;
a pressure cuff attachable to said support member in a position adjacent at least a portion of a periphery of the user's patella;
said support member having a plurality of connection points, said plurality of connection points located on at least one of opposite sides of the knee, above the knee, and below the knee
a plurality of connection members attachable between said pressure cuff and selective ones of said plurality of connection points on said support member; and
said pressure cuff being adjustable via connection of said plurality of connection members to said selective ones of said plurality of connection points to apply force to the patella in a plurality of directions.

2. The brace of claim 1 wherein said plurality of directions comprises a medial-lateral direction, an inferior-superior direction, and intermediate directions.

3. The brace of claim 1 further comprising at least one of said plurality of connection members having an adjustable length.

4. The brace of claim 1 wherein said support member is a sleeve, and further comprising:
a buttress adapted for positioning around at least a portion of the periphery of the patella; and
said buttress positioned between said pressure cuff and the patella.

5. The brace of claim 1 wherein said support member further comprises a first upper rigid arm connected by a first hinge to a first lower rigid arm, said first hinge located proximate the knee on a first side thereof, said first upper rigid arm extending above the knee and said first lower rigid arm extending below the knee.

6. The brace of claim 5 wherein said support member further comprises a second upper rigid arm connected by a second hinge to a second lower rigid arm, said second hinge located proximate the knee on a second side thereof, said second upper rigid arm extending above the knee and said second lower rigid arm extending below the knee.

7. The brace of claim 6 wherein said support member further comprises a sleeve in combination with said first and second upper and lower rigid arms, said sleeve adapted to locate about the knee of the user.

8. The brace of claim 7 further comprising:
at least one of said first and second upper and lower rigid arms having said plurality of connection points provided at spaced apart locations thereon;
at least one of said first and second hinges having an attachment portion;
said plurality of connection members attachable to at least one of said attachment portion of and said plurality of connection points; and
wherein attachment of said plurality of connection members to selective ones of said plurality of connection points varies said position at which said pressure cuff is held against the patella such that the direction in which said pressure cuff applies force to the patella is adjustable.

9. The brace of claim 8 further comprising:
a first one of said plurality of connection members attachable to said attachment portion of one of said first and second hinges;
a second one of said plurality of connection members attachable to respective ones of said plurality of connection points on said second upper rigid arm;
a third one of said plurality of connection members attachable to respective ones of said plurality of connection points on said second lower rigid arms; and
wherein attachment of said second and third ones of said plurality of connection members to selective ones of said plurality of connection points on said second upper and lower rigid arms varies said position at which said pressure cuff is held against the patella such that the direction in which said pressure cuff exerts force on the patella is adjustable.

10. The brace of claim 8 further comprising:
a first pair of said plurality of connection members attachable to respective ones of said plurality of connection points on said first upper and lower rigid arms, one of said first pair attachable to said first upper rigid arm and the other of said first pair attachable to said first lower rigid arm;
a second pair of said plurality of connection members attachable to respective ones of said plurality of connection points on said second upper and lower rigid arms, one of said second pair attachable to said second upper rigid arm and the other of said second pair attachable to said second lower rigid arm;
wherein attachment of each of said first and second pairs of said plurality of connection members to selective ones of said plurality of connection points varies said position at which said pressure cuff is held against the patella such that the direction in which said pressure cuff exerts force on the patella is adjustable.

11. The brace of claim 4 further comprising:
said buttress having a generally concave outer surface; and
said pressure cuff having a generally convex inner surface configured to mate with said generally concave outer surface of said buttress.

12. The brace of claim 11 further comprising said buttress having a relatively soft cushioned liner on an inner side thereof adjacent the patella, said liner conforming to the specific shape of the patella of a particular user to provide and enhanced fit when said pressure cuff exerts force against said patella via said buttress.

13. The brace of claim 1 further comprising:
said plurality of connection members attachable to said plurality of connection points via a hook and loop fastener system;
said plurality of attachment points forming either the hook or loop portion of the fastener system; and
a distal part of each of said plurality of connection members forming the other portion of the fastener system.

14. The brace of claim 13 further comprising at least one of said plurality of connection members having an adjustable length.

15. The brace of claim 1 further comprising:
at least one of said plurality of connection points having an aperture therein; and
at least one of said plurality of connection members having a distal part thereof with a protrusion configured to be received in said aperture to connect thereto.

16. The brace of claim 11 wherein said pressure cuff has an arcuate shape and further comprising:
said plurality of connection members being three connection members attached at each end of said arcuate shape and near a midpoint thereof, respectively;
one of said three connection members attached between said midpoint and said attachment portion first and hinge; and
another two of said three connection members attached between each end of said arcuate shape and respective ones of said first upper and lower rigid arms.

17. The brace of claim 11 wherein said pressure cuff has a circular or nearly circular shape and further comprising:
said plurality of connection members being four connection members attached at four generally equally spaced locations on said pressure cuff;
respective ones of said four connection members at said four locations attached to respective ones of said first and second upper and lower rigid arms.

18. A patella femoral brace comprising:
a sleeve configured fit snugly about a user's leg proximate the knee;
a pressure cuff exerting a force on at least a portion of a periphery of the user's patella;
said pressure cuff having one side attached to said sleeve, and upper and lower connection members extending from said one side;
said upper connection member attachable to an opposite side of said sleeve at multiple locations above the patella;
said lower connection member attachable to an opposite side of said sleeve at multiple locations below the patella;
wherein attachment of said upper and lower connection members to selective ones of said multiple locations above and below the patella enable said force exerted by said pressure cuff on the patella to be selectively applied in a plurality of directions.

19. The brace of claim 18 wherein said plurality of directions comprises a medial-lateral direction, an inferior-superior direction, and intermediate directions.

20. The brace of claim 18 further comprising a buttress member surrounding the patella, said buttress member held in position by said sleeve, and said buttress member is positioned intermediate the patella and said pressure cuff.

21. The brace of claim 20 further comprising:
a first upper rigid arm connected by a first hinge to a first lower rigid arm with said first hinge located proximate the knee; and
said first upper and lower rigid arms located at said opposite side of said sleeve.

22. The brace of claim 51 further comprising:
a second upper rigid arm connected by a second hinge to a second lower rigid arm with said first hinge located proximate the knee; and
said second upper and lower rigid arms located at said one side of said sleeve.

23. The brace of claim 18 further comprising at least one of said upper and lower connection members having an adjustable length.

24. The brace of claim 23 wherein attachment of said upper and lower connection members to said multiple locations is accomplished via a hook and loop fastener system, and further comprising:
said multiple locations formed by either the hook or loop portion of said fastener system;
said upper and lower connection members having a distal part thereof forming the other of the hook and loop fastener system; and
wherein each of said multiple locations and said adjustable length are enabled by said hook and loop fastener system.
